(19) United States
(12) Patent Application Publication
(10) Pub. No.: US 2005/0130201 A1
(43) Pub. Date: Jun. 16, 2005

(54) Splint-assisted enzymatic synthesis of polyribounucleotides

(75) Inventors: Michael Deras, San Diego, CA (US); Jeffrey A. Pleiss, San Francisco, CA (US); Stephen Scaringe, Lafayette, CO (US)

Correspondence Address: KALOW & SPRINGUT LLP, 488 MADISON AVENUE, 19TH FLOOR, NEW YORK, NY, 10022, US

(73) Assignee: DHARMACON, INC.

(21) Appl. No.: 10/965,547

(22) Filed: Oct. 13, 2004

Related U.S. Application Data
(60) Provisional application No. 60/511,493, filed on Oct. 14, 2003.

(51) Int. Cl. C12Q 1/68; C12P 19/34

(52) U.S. Cl. 435/6; 435/91.2

(57) ABSTRACT

The present invention comprises methods and compositions for splint-assisted enzymatic synthesis of polyribonucleotides using an RNA polymerizing enzyme. The invention provides ligating ribonucleotides comprising ligating a donor RNA molecule to an acceptor RNA molecule in the presence of RNA ligase and a splint, wherein the donor RNA molecule is comprised of at least one nucleotide and a ligation linker moiety, the acceptor RNA molecule is comprised of at least one nucleotide and a ligation linker moiety and the splint is comprised of a polyribonucleotide. The invention also provides splints for use in splint-assisted enzymatic synthesis using an RNA polymerizing enzyme.

### FIELD OF THE INVENTION

The present invention relates to the field of enzymatic RNA synthesis.

### BACKGROUND

Currently, the most widely employed methods to produce RNA oligonucleotides or polynucleotides are enzymatic transcription and chemical synthesis. Transcription permits RNA sequences and in certain cases RNAs of lengths from approximately 20 bases into the thousands of bases to be prepared. However, polymerase catalyzed transcription often requires laborious design of DNA templates, does not permit the incorporation of non-canonical modifications, and suffers from inconsistency when sequence effects interfere with enzyme processivity.

The chemical synthesis of RNA provides the ability to synthesize well-defined RNAs. The principal advantages of chemical synthesis are: (1) a number of modifications can be site-specifically incorporated; (2) modifications can include deoxy bases, natural bases, e.g. pseudouridine, or unnatural ribonucleosides, e.g. 5-bromo-uridine; (3) RNAs as short as 2-3 bases are easily synthesized; and (4) in theory, chemical synthesis is a more reliable method because it is less susceptible to primary sequence effects. Unfortunately, known chemical methods have length limitations such that synthesis of RNAs as long as 100 or more base pairs can be costly, inefficient, offer relatively low yield, and is laborious.

One chemical synthesis method is based on ligase technologies. For example, for several years, oligo ligations have been performed using T4 DNA ligase (Bain, J. D., and Switzer, C. (1992) Nucleic Acids Res 20, 4372). The substrate for these reactions is a complex of two RNA oligos annealed to a third DNA oligo (the splint) that bridges the splice site. This complex is a poor substrate for ligation, and the enzyme must be used in stoichiometric amounts.

An alternative to using a DNA ligase is to use an RNA ligase. Early work using T4 RNA ligase was done in the labs of Ohlike Uhlenbeck and Richard Gumport. (Romaniuk, P. J., and UhLenbeck, O. C. (1983) Methods Enzymol 100, 52-92-5; Krug, M., and Uhlenbeck, O. C. (1982) Biochemistry 21, 1858-64; Meyhack, B., Pace, B., Uhlenbeck, O. C., and Pace, N. R. (1978) Proc Natl Acad Sci USA 75, 3045-9; Uhlenbeck, O. C., and Cameron, V. (1977) Nucleic Acids Res 4, 85-98). According to this method, the native substrate for the enzyme is the cleaved anticodon loop of tRNA molecules that results from pre-tRNA processing. The enzyme requires ATP, a 5′-phosphate on the downstream or ‘donor’ substrate, and that the sequence of the splice junction be single stranded.

Most preliminary research using T4 RNA focused on nucleotide coupling reactions. However, several complications were observed during these investigations: (1) the reaction suffered from the slow kinetics of three substrates coming together on the enzyme; (2) the first steps are enzyme adenylation and subsequent adenyl-transfer to the donor; (3) in the absence of upstream or ‘acceptor’ oligo, irreversible release of an adenylated donor occurs (depleting the available pool); and (4) T4 RNA ligase does not equally recognize all nucleotides as acceptors or donors.

Thus, there is a need in the art of RNA synthesis for economical and efficient methods of making oligoribonucleotides longer than fifty bases, and up to and exceeding one hundred bases, that retain the advantages of chemical synthesis.

### SUMMARY OF THE INVENTION

The present invention is directed to compositions and methods for splint-assisted enzymatic synthesis of oligoribonucleotides.

According to a first embodiment, the present invention provides a method of ligating ribonucleotides comprising: ligating a donor RNA molecule to an acceptor RNA molecule in the presence of RNA ligase and a splint, wherein the donor RNA molecule is comprised of at least one nucleotide and a ligation linker moiety, the acceptor RNA molecule is comprised of at least one nucleotide and a ligation linker moiety and the splint is comprised of a polyribonucleotide.

According to a second embodiment, the present invention provides a splint for use in ligating RNA molecules, comprising a polyribonucleotide, wherein said polyribonucleotide has at least one orthoester modified nucleotide base.

Through the use of the present invention, splint-assisted oligoribonucleotide synthesis can be performed to synthesize RNAs. The advantages of the present invention include the ability to ligate RNA molecules using an RNA ligase.

For a better understanding of the present invention together with other and further advantages and embodiments, reference is made to the following description taken in conjunction with the examples, the scope of the which is set forth in the appended claims.

### BRIEF DESCRIPTION OF THE FIGURES

The preferred embodiments of the present invention have been chosen for purposes of illustration and description but are not intended to restrict the scope of the invention in any way. The benefits of the preferred embodiments of certain aspects of the invention are shown in the accompanying figures, wherein:

FIG. 1 illustrates the general structure of the most preferred orthoester modification, a 2′-O-bis(2-hydroxyethoxy)methyl orthoester, also referred to as a 2′-ACE RNA.

FIG. 2 illustrates the initial system used in the initial ligation studies.

FIG. 3A illustrates the results of ATP titration of a ligation reaction at 0.4 U/microliter ligase.

FIG. 3B illustrates the results of ATP titration of a ligation reaction at 0.8 U/microliter ligase.

FIG. 4 illustrates a ligase titration.

FIG. 5 illustrates the effect of annealing on reaction outcome.

FIG. 6 illustrates second generation splints designed to test the effect of B:splint pairing on reaction outcome.

FIG. 7A illustrates the results of AMP titration of a ligation reaction.

FIG. 7B illustrates the results of pyrophosphate (PPᵢ) titration of a ligation reaction.

FIG. 8A illustrates reaction progress with 3′-blocked B substrates.

FIG. 8B illustrates reaction progress with 3′-blocked B substrates.

FIG. 9A illustrates ligation progress using either 2′-OH or 2′-ACE B substrate.

FIG. 9B illustrates ligation progress using either 2′-OH or 2′-ACE B substrate.

FIG. 10 illustrates variations in ligation linker length.

FIG. 11A1 illustrates the effect of variations in the length of A and B ligation linkers.

FIG. 11A2 illustrates the effect of variations in the length of A and B ligation linkers.

FIG. 11B1 illustrates the effect of variations in the length of A and B ligation linkers.

FIG. 11B2 illustrates the effect of variations in the length of A and B ligation linkers.

FIG. 11C illustrates the effect of variations in the length of longer 2′-ACE B ligation linkers.

FIG. 12A illustrates polypyrimidine ligation linkers as potential substrates.

FIG. 12B illustrates results of experiments using polypyrimidine ligation linkers.

FIG. 12C illustrates results of experiments using polypyrimidine ligation linkers with 2′-ACE modifications.

FIG. 13 illustrates the effect of varying RNA concentration.

FIG. 14 illustrates the results of a stoichiometry study.

### DETAILED DESCRIPTION

Unless stated otherwise, the following terms and phrases have the meanings provided below:

Alkyl

The term “alkyl” refers to a hydrocarbyl moiety that can be saturated or unsaturated, and substituted or unsubstituted. It may comprise moieties that are linear, branched, cyclic and/or heterocyclic, and contain functional groups such as ethers, ketones, aldehydes, carboxylates, etc.

Exemplary alkyl groups include but are not limited to substituted and unsubstituted groups of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl and alkyl groups of higher number of carbons, as well as 2 -methylpropyl, 2-methyl-4-ethylbutyl, 2,4-diethylpropyl, 3-propylbutyl, 2,8-dibutyldecyl, 6,6-dimethyloctyl, 6-propyl-6-butyloctyl, 2-methylbutyl, 2-methylpentyl, 3-methylpentyl, and 2-ethylhexyl. The term alkyl also encompasses alkenyl groups, such as vinyl, allyl, aralkyl and alkynyl groups.

Substitutions within an alkyl group can include any atom or group that can be tolerated in the alkyl moiety, including but not limited to halogens, sulfurs, thiols, thioethers, thioesters, amines (primary, secondary, or tertiary), amides, ethers, esters, alcohols and oxygen. The alkyl groups can by way of example also comprise modifications such as azo groups, keto groups, aldehyde groups, carboxyl groups, nitro, nitroso or nitrile groups, heterocycles such as imidazole, hydrazino or hydroxylamino groups, isocyanate or cyanate groups, and sulfur containing groups such as sulfoxide, sulfone, sulfide, and disulfide.

Further, alkyl groups may also contain hetero substitutions, which are substitutions of carbon atoms, by for example, nitrogen, oxygen or sulfur. Heterocyclic substitutions refer to alkyl rings having one or more heteroatoms. Examples of heterocyclic moieties include but are not limited to morpholino, imidazole, and pyrrolidino.

2′-O-alkyl Modified Nucleotide

The phrase “2′-O-alkyl modified nucleotide” refers to a nucleotide unit having a sugar moiety, for example a deoxyribosyl moiety that is modified at the 2′ position such that an oxygen atom is attached both to the carbon atom located at the 2′ position of the sugar and to an alkyl group.

Amine and 2′ Amine Modified Nucleotide

The term “amine” refers to moieties that can be derived directly or indirectly from ammonia by replacing one, two, or three hydrogen atoms by other groups, such as, for example, alkyl groups. Primary amines have the general structures RNH₂ and secondary amines have the general structure R₂NH. The phrase “2′amine modified nucleotide” refers to a nucleotide unit having a sugar moiety that is modified with an amine or nitrogen containing group attached to the 2′ position of the sugar.

The term amine includes, but is not limited to methylamine, ethylamine, propylamine, isopropylamine, aniline, cyclohexylamine, benzylamine, polycyclic amines, heteroatom substituted aryl and alkylamines, dimethylamine, diethylamine, diisopropylamine, dibutylamine, methylpropylamine, methylhexylamine, methylcyclopropylamine, ethylcylohexylamine, methylbenzylamine, methycyclohexylmethylamine, butylcyclohexylamine, morpholine, thiomorpholine, pyrrolidine, piperidine, 2,6-dimethylpiperidine, piperazine, and heteroatom substituted alkyl or aryl secondary amines.

Complementary

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes.

Perfect complementarity or 100% complementarity refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. Substantial complementarity refers to polynucleotide strands exhibiting 79% or greater complementarity, excluding regions of the polynucleotide strands, such as overhangs, that are selected so as to be noncomplementary. Thus, for example, two polynucleotides of 29 nucleotide units each, wherein each comprises a di-dT at the 3′ terminus such that the duplex region spans 27 bases, and wherein 26 of the 27 bases of the duplex region on each strand are complementary, are substantially complementary since they are 96.3% complementary when excluding the di-dT overhangs.

Conjugate and Terminal Conjugate

The term “conjugate” refers to a molecule or moiety that alters the physical properties of a polynucleotide such as those that increase stability and/or facilitate uptake of double stranded RNA by itself. A “terminal conjugate” may be attached directly or through a conjugate linker to a 3′ and/or 5′ end of a polynucleotide or double stranded polynucleotide. An internal conjugate may be attached directly or indirectly through a conjugate linker to a base, to the 2′ position of the ribose, or to other positions that do not interfere with Watson-Crick base pairing, for example, 5-aminoallyl uridine.

In a double stranded polynucleotide, one or both 5′ ends of the strands of polynucleotides comprising the double stranded polynucleotide can bear a conjugate, and/or one or both 3′ ends of the strands of polynucleotides comprising the double stranded polynucleotide can bear a conjugate.

Conjugates may, for example, be amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of conjugates also include steroids, such as cholesterol, phospholipids, di- and tri-acylglycerols, fatty acids, hydrocarbons that may or may not contain unsaturation or substitutions, enzyme substrates, biotin, digoxigenin, and polysaccharides. Still other examples include thioethers such as hexyl-S-tritylthiol, thiocholesterol, acyl chains such as dodecandiol or undecyl groups, phospholipids such as di-hexadecyl-rac-glycerol, triethylammonium 1,2-di-O-hexadecyl-rac-glycero-3-H-phosphonate, polyamines, polyethylene glycol, adamantane acetic acid, palmityl moieties, octadecylamine moieties, hexylaminocarbonyl-oxycholesterol, farnesyl, geranyl and geranylgeranyl moieties.

Conjugates can also be detectable labels. For example, conjugates can be fluorophores. Conjugates may include fluorophores such as TAMRA, BODIPY, Cyanine derivatives such as Cy3 or Cy5 Dabsyl, or any other suitable fluorophore known in the art.

A conjugate may be attached to any position on the terminal nucleotide that is convenient and that does not substantially interfere with the desired activity of the polynucleotide(s) that bear it, for example the 3′ or 5′ position of a ribosyl sugar. A conjugate substantially interferes with the desired activity of an RNA if it adversely affects its functionality such that the ability of the RNA to carry out it intended function is reduced by greater than 80%.

Conjugate Linker

A “conjugate linker” is a moiety that attaches two or more other moieties to each other such as a nucleotide and its conjugate. A conjugate linker may be distinguished from a conjugate in that while a conjugate increases the stability and/or ability of a molecule to be taken up by a cell, or imparts another attribute to the molecule, a conjugate linker merely attaches a conjugate to the molecule that is to be introduced into the cell.

By way of example, conjugate linkers can comprise modified or unmodified nucleotides, nucleosides, polymers, sugars and other carbohydrates, polyethers such as, for example, polyethylene glycols, polyalcohols, polypropylenes, propylene glycols, mixtures of ethylene and propylene glycols, polyalkylamines, polyamines such as spermidine, polyesters such as poly(ethyl acrylate), polyphosphodiesters, and alkylenes. An example of a conjugate and its linker is cholesterol-TEG-phosphoramidites, wherein the cholesterol is the conjugate and the tetraethylene glycol and phosphate serve as conjugate linkers. The phrase “conjugate linker” should be distinguished from the term “linker,” which is employed herein to refer to the portion of a donor or acceptor RNA that does not anneal with a splint in splint-assisted RNA ligation.

Deoxynucleotide

The term “deoxynucleotide” refers to a nucleotide or polynucleotide lacking an OH group at the 2′ and/or 3′ position of a sugar moiety. Instead it has a hydrogen bonded to the 2′ and/or 3′ carbon. Within an RNA molecule that comprises one or more deoxynucleotides, “deoxynucleotide” refers to the lack of an OH group at the 2′ position of the sugar moiety, having instead a hydrogen bonded directly to the 2′ carbon.

Deoxyribonucleotide

The terms “deoxyribonucleotide” and “DNA” refer to a nucleotide or polynucleotide comprising at least one sugar moiety that has an H, rather than an OH, at its 2′ and/or 3′position.

Duplex Region

The phrase “duplex region” refers to the region in two complementary or substantially complementary polynucleotides that form base pairs with one another, either by Watson-Crick base pairing or any other manner that allows for a stabilized duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” has 19 base pairs. The remaining base pairs may, for example, exist as 5′ and 3′ overhangs. Further, within the duplex region, 100% complementarity is not required; substantial complementarity is allowable within a duplex region. Substantial complementarity refers to 79% or greater complementarity. For example, a mismatch in a duplex region consisting of 19 base pairs results in 94.7% complementarity, rendering the duplex region substantially complementary.

Halogen

The term “halogen” refers to an atom of either fluorine, chlorine, bromine, iodine or astatine. The phrase “2′ halogen modified nucleotide” refers to a nucleotide unit having a sugar moiety that is modified with a halogen at the 2′ position, attached directly to the 2′ carbon.

Internucleotide Linkage

The phrase “internucleotide linkage” refers to the type of bond or linkage that is present between two nucleotide units in a polynucleotide and may be modified or unmodified. The phrase “modified internucleotide linkage” includes all modified internucleotide linkages now known in the art or that come to be known and that, from reading this disclosure, one skilled in the art will conclude is useful in connection with the present invention. Internucleotide linkages may have associated counterions, and the term is meant to include such counterions and any coordination complexes that can form at the internucleotide linkages. A modified internucleotide linkage can serve as a nucleus uptake modification.

Modifications of internucleotide linkages include, but are not limited to, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedinethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that can be saturated or unsaturated and/or substituted and/or contain heteroatoms, linkages with morpholino structures, amides, polyamides wherein the bases can be attached to the aza nitrogens of the backbone directly or indirectly, and combinations of such modified internucleotide linkages within a polynucleotide. The term “thio modified internucleotide linkage” includes any internucleotide linkage that comprises at least one sulfur atom.

Ligation Linker

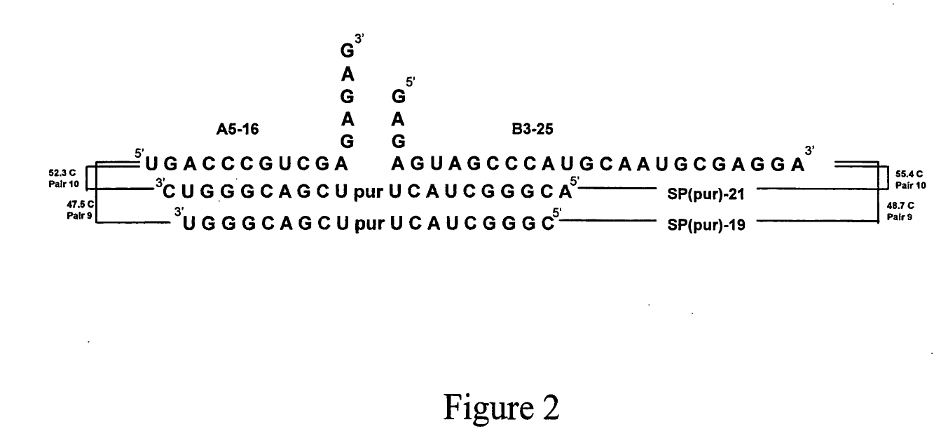

The phrase “ligation linker” refers to the 3′ region of the acceptor RNA or the 5′ region of a donor RNA that does not anneal to the splint in splint-assisted ligation. Examples of linkers can be found in FIGS. 2, 6, 10 and 12A.

Nucleotide

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH2, NHR, NR2, or CN, wherein R is an alkyl moiety as defined herein. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

Modified bases refer to nucleotide bases such as, for example, adenine, guanine, cytosine, thymine, and uracil, xanthine, inosine, and qucuosine that have been modified by the replacement or addition of one or more atoms or groups. Some examples of types of modifications that can comprise nucleotides that are modified with respect to the base moieties, include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. The term nucleotide is also meant to include what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group.

Further, the term nucleotide also includes those species that have a detectable label, such as for example a radioactive or fluorescent moiety, or mass label attached to the nucleotide.

Nucleotide Unit

The phrase “nucleotide unit” refers to a single nucleotide residue and is comprised of a modified or unmodified nitrogenous base, a modified or unmodified sugar, and a modified or unmodified moiety that allows for linking of two nucleotides together or a nucleotide to a conjugate that precludes further linkage. The single nucleotide residue may be in a polynucleotide. Thus, a polynucleotide having 27 bases has 27 nucleotide units.

Orthoester Protected and Orthoester Modified

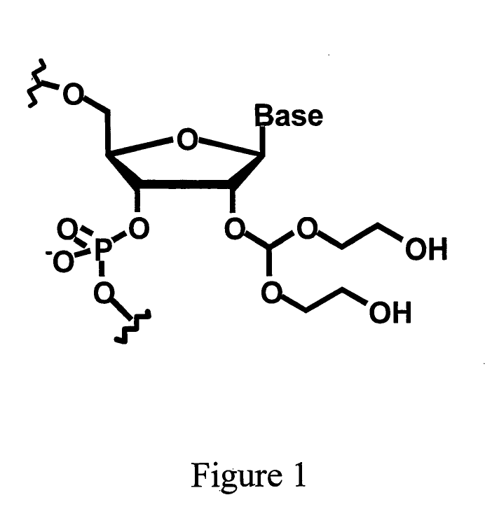

The phrases “orthoester protected” and “orthoester modified” refer to modification of a sugar moiety within a nucleotide unit with an orthoester. Preferably, the sugar moiety is a ribosyl moiety. In general, orthoesters have the structure RC(OR′)₃ wherein each R′ can be the same or different, R can be an H, and wherein the underscored C is the central carbon of the orthoester. The orthoesters of the present invention are comprised of orthoesters wherein a carbon of a sugar moiety in a nucleotide unit is bonded to an oxygen, which is in turn bonded to the central carbon of the orthoester. To the central carbon of the orthoester are, in turn, bonded two oxygens, such that in total three oxygens bond to the central carbon of the orthoester. These two oxygens bonded to the central carbon (neither of which is bonded to the carbon of the sugar moiety) in turn, bond to carbon atoms that comprise two moieties that can be the same or different. For example, one of the oxygens can be bound to an ethyl moiety, and the other to an isopropyl moiety. In one example, R can be an H, one R′ can be a ribosyl moiety, and the other two R′ moieties can be 2-ethyl-hydroxyl moieties. The foregoing is also the definition of an “orthoester moiety.” Orthoesters can be placed at any position on the sugar moiety, such as, for example, on the 2′, 3′ and/or 5′ positions. Preferred orthoesters, and methods of making orthoester protected polynucleotides, are described in U.S. Pat. Nos. 5,889,136 and 6,008,400, each herein incorporated by reference in its entirety. An example of an orthoester, or orthoester moiety, is 2′-O-bis(2-hydroxyethoxy)methyl orthoester, depicted in FIG. 1, which is also the most preferred orthoester.

Polynucleotide

The term “polynucleotide” refers to polymers of nucleotides, and includes but is not limited to DNA, RNA, DNA/RNA hybrids including polynucleotide chains of regularly and irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides wherein the attachment of various entities or moieties to the nucleotide units at any position are included.

Polyribonucleotide

The term “polyribonucleotide” refers to a polynucleotide comprising two or more modified or unmodified ribonucleotides and/or their analogs. The term “polyribonucleotide” is used interchangeably with the term “oligoribonucleotide.”

Ribonucleotide and Ribonucleic Acid

The term “ribonucleotide” and the phrase “ribonucleic acid” (RNA), refer to a modified or unmodified nucleotide or polynucleotide comprising at least one ribonucleotide unit. A ribonucleotide unit comprises an oxygen attached to the 2′ position of a ribosyl moiety that has a nitrogenous base attached in N-glycosidic linkage at the 1′ position of a ribosyl moiety, and a moiety that either allows for linkage to another nucleotide or precludes linkage.

Spacer

The term “spacer” refers to a region of a splint that occurs opposite the splice site of a donor RNA molecule and an acceptor RNA molecule, and is unable to hybridize or anneal to either the donor RNA molecule or the acceptor RNA molecule under ligation reaction conditions. An example of a spacer is one or more purine nucleotide bases. However, as spacer may be comprised of any material that does not hybridize or anneal to either the donor RNA molecule or the acceptor RNA molecule.

Stabilized

The term “stabilized” refers to the ability of a dsRNA to resist degradation while maintaining functionality and can be measured in terms of its half-life in the presence of, for example, biological materials such as serum. The half-life of an RNA in, for example, serum refers to the time taken for the 50% of the RNA to be degraded.

Wherever a range of values is provided in this disclosure, each intervening value, unless the context dictates otherwise, is encompassed within the invention. Further, it is understood that the invention includes, for each value, tenths of the lower limit indicated, unless the context clearly dictates otherwise. The invention also includes the upper and lower limit of the stated range, unless otherwise indicated. The upper and lower limits of smaller ranges may independently be included in the smaller ranges. Where the stated range includes one or both of the limits, ranges excluding either or both of those included limits are also included in the invention.

### PREFERRED EMBODIMENTS

The present invention will now be described in connection with preferred embodiments. These embodiments are presented to aid in an understanding of the present invention and are not intended, and should not be construed, to limit the invention in any way. All alternatives, modifications and equivalents that may become apparent to those of ordinary skill upon reading this disclosure are included within the spirit and scope of the present invention.

This disclosure is not a primer on the synthesis of oligoribonucleotides. Basic concepts known to those skilled in the art have not been set forth in detail.

According to a first embodiment, the present invention provides a method of ligating ribonucleotides. The method comprises ligating a donor RNA molecule to an acceptor RNA molecule in the presence of RNA ligase and a splint, wherein the donor RNA molecule is comprised of at least one nucleotide and a ligation linker moiety, the acceptor RNA molecule is comprised of at least one nucleotide and a ligation linker moiety and the splint is comprised of a polyribonucleotide.

The donor RNA molecule may comprise one or more modified nucleotides. The one or more modified nucleotides may bear any modification known in the art, or any combination of modifications known in the art. Preferably, the modification comprises a stabilizing modification. A stabilizing modification is a modification that confers resistance to nucleases and/or chemical degradation. Preferable modifications also include those modifications that affect the flexibility of the RNA molecule. More preferably, the modification reduces the flexibility of the donor RNA molecule.

The inventive splint may further comprise at least one modified nucleotide. Preferred modified nucleotides for the splint include the same modified nucleotides as for the donor RNA molecule. As is the case with the donor RNA molecule, modifications that confer resistance to nucleases and/or chemical degradation (such as, for example, phosphorothioate modified nucleotides, 2′-orthoester modified nucleotides, and 2′-O-methyl modified nucleotides), as well as those that reduce the flexibility of the splint (such as 2′-orthoester modified nucleotides), are desirable. The splint is designed such that it has two regions of complementarity to the RNA molecules to be ligated. One region of the splint is substantially complementary to the donor RNA molecule but not the donor RNA molecule's ligation linker, a second region of the splint is substantially complementary to the acceptor RNA molecule but not the acceptor RNA molecule's ligation linker. The splint region that is substantially complementary to the acceptor RNA does not overlap with the splint region that is substantially complementary to the donor RNA. In this way, the splint can anneal with the donor RNA and the acceptor RNA, bringing the donor RNA and acceptor RNA in close proximity so as to facilitate ligation.

Without wishing to be bound by any one theory, it is postulated that a primary advantage of the splint is the ability to improve ligation reaction kinetics by assisting in forming a ternary complex between the donor RNA, the acceptor RNA, and the splint, favorably influencing the kinetics of the reaction by effectively increasing the concentration of productive conformations between the donor RNA and the acceptor RNA at the active site of the RNA ligase. It is postulated that modifications that decrease the probability of intramolecular secondary and/or tertiary structure formations in the splint and the donor RNA molecule that would be unfavorable to the ligation reaction, such as, for example, 2′ orthoester modifications, have a beneficial effect on the reaction kinetics, at least in part due to more efficient ternary complex formation. Modifications other than those that decrease the probability of secondary and/or tertiary structure formation in the splint and the donor RNA molecule are, of course, allowed, and may be desirable under certain circumstances such as, for example, when ligating modified RNA molecules such as, for example, modified ribosomal RNAs, modified tRNA, modified ribozymes, longer RNAs containing pseudouridine or inosine and RNAs that are desired to possess some measure of nuclease resistance and/or resistance to chemical degradation.

Additionally, it is postulated that reduction in flexibility of the donor RNA molecule can improve ligation yields in certain cases. Reduction in flexibility of a donor RNA molecule can result in improved ligation due to a reduced capacity of the donor RNA molecule to form secondary and/or tertiary structures that may be detrimental to the formation of a donor/acceptor/splint complex that promotes the formation of a desired ligation product A donor/acceptor/splint complex is a ternary complex that results in the 5′ terminus of the donor RNA molecule and the 3′ terminus of the acceptor RNA molecule being in close proximity to facilitate the ligation reaction. Formation of the ternary complex is in part dependent upon the ability of the donor RNA molecule and the acceptor RNA molecule to anneal to the splint.

Certain modifications, such as orthoester modifications, can confer nuclease resistance while at the same time reducing the flexibility of the RNA molecule. Preferably, at least one modified nucleotide is present and the at least one modified nucleotide is an orthoester modified nucleotide. More preferably, the orthoester modified nucleotide is an orthoester moiety bonded to a 2′ carbon of said at least one modified nucleotide of the donor RNA. Most preferably, each nucleotide of the donor RNA has an orthoester bonded to the 2′ carbon of each ribosyl moiety. Preferred orthoesters, and methods of making orthoester protected polyribonucleotides, are described in U.S. Pat. Nos. 5,889,136 and 6,008,400, each herein incorporated by reference in its entirety. The most preferred orthoester is a 2′-O-bis(2-hydroxyethoxy)methyl orthoester, depicted in FIG. 1. A polyribonucleotide having a 2′-O-bis(2-hydroxyethoxy)methyl orthoester is also referred to herein as a “2′-ACE RNA.”

The donor RNA molecule, the acceptor RNA molecule and/or the splint may be comprised of one or more modified nucleotides. Preferred modified nucleotides for all three molecules include modifications that confer resistance to nucleases and/or chemical degradation. Preferred modified nucleotides for the donor RNA molecule and the splint include modifications that disfavor the formation of intramolecular secondary and/or tertiary structures. Preferably, the acceptor RNA molecule comprises a first modified nucleotide, and the donor RNA molecule comprises a second modified nucleotide that is different from the first modified nucleotide. Preferably, modifications of the acceptor RNA molecule do not decrease the flexibility of the acceptor RNA molecule.

The splint may further comprise a spacer. The spacer is preferably comprised of one or more purine ribonucleotides opposite the splice junction of the donor RNA molecule and the acceptor RNA molecule. Preferably, neither the donor RNA molecule nor the acceptor RNA molecule can base pair with the spacer.

Preferably, the splint comprises a polyribonucleotide having a blocked 3′ terminus. Without wishing to be bound by any particular theory, it is postulated that using a splint with a blocked 3′ terminus can help reduce the probability of undesired ligation products, such as concatamers, by, in effect, preventing the splint from acting as an acceptor molecule. Many methods are known in the art for blocking the 3′ terminus of a polyribonucleotide. Preferably, an inverted deoxythymidine is used at the 3′ terminus (3′-idT) of the splint.

Preferably, the ligation reaction proceeds to at least 80% completion. More preferably, the ligation reaction proceeds to at least 85% completion. By percent completion is meant the percent to which the acceptor RNA and the donor RNA are ligated to one another to form the desired ligation product. Percent completion is based upon the amount of limiting substrate. Preferably, the ration of acceptor RNA (A) to splint to donor RNA (B) is 1:1.25:1.5.

Preferably, the acceptor RNA molecule comprises a 3′terminal —OH, and preferably the donor RNA molecule comprises a 5′ terminal phosphate moiety.

The donor RNA and the acceptor RNA each comprise a ligation linker. The ligation linker of the donor RNA is at its 3′ end. The ligation linker of the acceptor RNA is at its 5′ end. Preferably, the length of the ligation linker of the acceptor RNA is greater than or equal to four nucleotide bases. Preferably, the length of the ligation linker of the donor RNA molecule is greater than or equal to two nucleotide bases. More preferably, the length of the ligation linker of the donor RNA molecule is eleven to twelve nucleotide bases. The ligation linker of the donor RNA molecule and the ligation linker of the acceptor RNA molecule preferably should not be able to form a duplex with the splint under the ligation reaction conditions employed. Either or both ligation linkers may comprise any modifications known in the art, including modified internucleotide linkages.

The donor and the acceptor RNA molecules should each anneal to the splint under the ligation reaction conditions in order to form duplexes with the splint. Preferably, the donor RNA molecule and the acceptor RNA molecule each have a Tm with respect to the splint of at least 45 degrees Centigrade.

Preferably, the ligation reaction is carried out in the presence of a pyrophosphatase. Pyrophosphate, a product of the ligation reaction, is a product inhibitor. Preferably, any pyrophosphatase used is nuclease-free.

In a second embodiment, the present invention provides a splint for use in ligating RNA molecules. The splint comprises a polyribonucleotide, wherein said polyribonucleotide has at least one orthoester modified nucleotide base. Additionally, the splint preferably comprises a spacer. Preferably, the splint comprises at least one orthoester modified nucleotide base wherein the orthoester modified nucleotide base is a 2′ orthoester modified nucleotide base. The splint of this second embodiment can encompass all the features recited in connection with the splint of the first embodiment.

The acceptor and/or donor RNA of both the first and second embodiments may also contain stabilization modifications such as orthoesters, 2′-O-methyl groups, fluoro groups and stabilizing conjugates as described in commonly assigned co-pending application entitled Stabilized Polynucleotides for use in RNA Interference, filed Apr. 2, 2003, U.S. Ser. No. 10/406,908, the entire disclosure of which is herein incorporated by reference.

The acceptor, donor, and the splint of both the first and second embodiments may be synthesized by any method that is now known or that comes to be known for synthesizing RNA molecules and that from reading this disclosure, one skilled in the art would conclude would be useful in connection with the present invention. For example, one may use methods of chemical synthesis such as methods that employ Dharmacon, Inc.'s proprietary ACE® technology. Alternatively, one could also use template dependant synthesis methods.

The acceptor, donor, and the splint of both the first and second embodiments may also contain stabilization modifications as described in connection with the first embodiment. Further, the RNAs may be synthesized in the same manner as described in connection with the first embodiment.

Certain fundamental advantages of the present invention, including the first and second embodiments, as well as embodiments described below and in the Examples, can be understood with reference to FIGS. 2 through 14.

Having described the invention with a degree of particularity, examples will now be provided. These examples are not intended to and should not be construed to limit the scope of the claims in any way. Although the invention may be more readily understood through reference to the following examples, they are provided by way of illustration and are not intended to limit the present invention unless specified.

### EXAMPLES

In investigating spacer composition, all splints used a purine ribonucleoside spacer opposite of the splice junction. Neither A nor B could base pair with this spacer, but the helical stacking of the splint itself was preserved.

### EXAMPLE 1

### Initial Ligation System

Oligonucleotides were prepared using the 2′-ACE method on modified Applied Biosystems 380B synthesizers, using standard amidites. All HPLC was performed on Waters chromatography systems with DNA-PAC anion exchange columns at 55° C. Buffer A: 5 mM sodium perchlorate, 10 mM Tris, 5 M urea, 2% acetonitrile, pH 8.0. Buffer B: 300 mM NaClO₄, 10 mM Tris, 5 M urea, 2% acetonitrile, pH 8.0. The gradient was (1.5 mL/min) 35-85% B from 3′-25′. Detection was at 260 mm.

T4 RNA Ligase was purchased from NEB (part M0204L). ATP (part A2,620-9) was purchased from Aldrich, while AMP (part 1752), inorganic pyrophosphate (part P-9146), and inorganic pyrophosphatase (part I-1643) were from Sigma. All other reagents and buffers were purchased from standard commercial sources.

Calculations of Tₘ for A:splint and B:splint pairings were performed using the Breslauer calculation found at: http://alces.med.umn.edu/rawtm.htl.(Breslauer, K J., Frank, R., Blocker, H., and Marky, L. A. (1986) Proc Natl Acad Sci USA 83, 3746-50) These numbers were only used to provide relative melting points, as the algorithm is based on DNA, not RNA data.

For all the experiments of this initial system, the splint consisted of a 2′-ACE protected oligo with a purine spacer opposite the splice site. To this splint, both the acceptor (A) and donor (B) oligos were annealed. The B oligo was 5′-phosphorylated. Unless specifically mentioned as in Section C.7, A and B were in the deprotected 2′-OH form. The ratio of A:splint:B was held constant at 1:1.25:1.5 except where indicated otherwise, in order to assure that all A was bound to splint, and all A:splint complex was bound to B. The length of the single stranded splice junction of A and B was held constant except as described in experiments in connection with single stranded length and ligation linker composition. The A ligation linker (3′-end of acceptor) was 5 bases long, and the B ligation linker (5′-end of donor) was 3 bases long. A 41-mer control oligo was chemically synthesized to establish the HPLC retention of the desired product. 120 μL reactions were set up with the following components unless otherwise indicated:

TABLE 1
Stoichiometry of Ligase Reactions.

| Component | Stoichiometry | Final Concentration | |
|---|---|---|---|
| A, acceptor | 6 nmol, 1 eq | 50 | μM |
| Splint | 7.5 nmol, 1.25 eq | 63 | μM |
| B, donor | 9 nmol, 1.5 eq | 75 | μM |
| Tris.Cl, pH 7.8 | | 50 | mM |
| MgCl₂ | | 10 | mM |
| DTT | | 10 | mM |
| ATP | 90 nmol, 10 eq rel to donor B | 250 | μM |
| Ligase | 9.6 U/μL | 0.8 | U/μL |

All components except enzyme were vortexed and microfuged before ligase was added and gently mixed All reactions were run at ambient temperature. Time points were taken at 0.5, 1, 3 and 24 hrs by removing 30 μL aliquots and thoroughly mixing each into 130 μL of 7 M urea in HPLC autosampler vials.

The first reactions were performed using the combination of acceptor, donor, and splint oligos as shown in FIG. 2. The original goal was to titrate ATP concentration at several RNA concentrations. RNA concentrations were 50 or 100 μM A, 0.25, 1, 5, or 10 eq. of ATP relative to B, and either splint 21 or 19. Enzyme concentration was 0.2 U/μL.

### EXAMPLE 2

### Titrating ATP Concentration

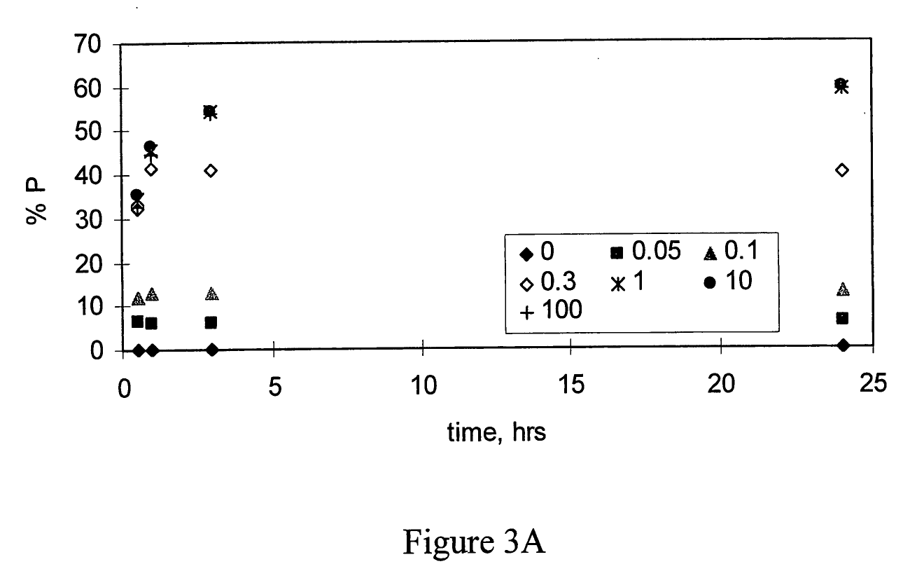

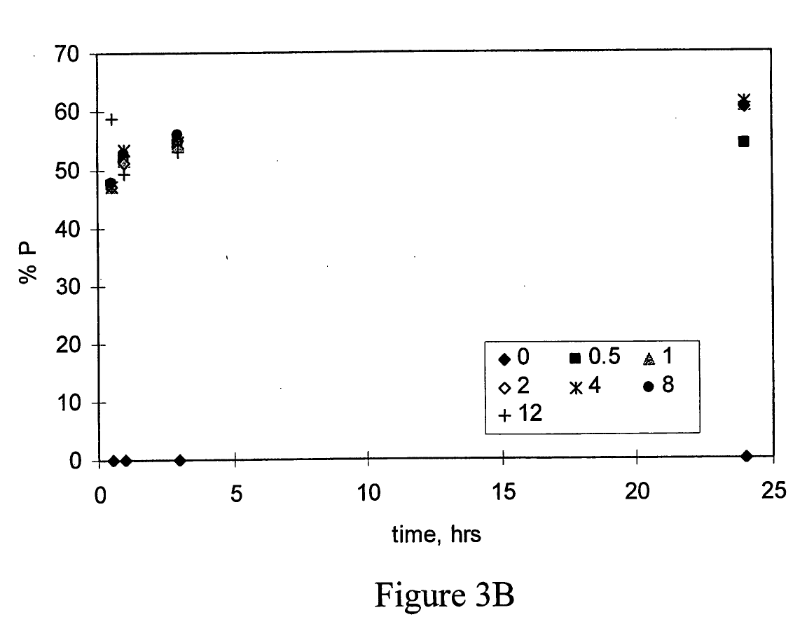

In order to optimize ligation reaction conditions, reactions were run in which ATP concentrations were titrated at various concentrations of donor RNA, acceptor RNA, and splint polyribonucleotide. Two splints were used in these optimization reactions, denoted splint 21 and splint 19. These splints are illustrated in FIG. 2. The results of the optimization reactions are shown in FIGS. 3A and 3B.

At 50 μM A, reactions run with splint 21 cleanly afforded a single product that co-eluted with 41-mer control. Reactions with splint 19 gave a mixture of products: the desired product in 66%, and minor products of 7%, 5%, and 22%. ATP concentration at or above 1 eq had no effect on this outcome. At 100 μM A, reactions using either splint were observed to provide multiple products in roughly the same ratio. Again, ATP concentration had no effect. In all cases, ligase concentration was too low to be certain that reactions could reach completion faster than the natural degradation of the enzyme. With these results in hand, new splints were designed and the results are discussed in connection with the splint design experiments described herein below. Meanwhile, additional ATP titrations were performed using only splint 21 and higher concentrations of ligase.

Ligations using A, B, and splint 21 (50 μM A) were performed at several equivalents of ATP (relative to B). (FIGS. 3A and 3B) All ligations cleanly afforded the desired product Final ligase concentration was 0.4 or 0.8 U/μL. At all concentrations of enzyme, the results were identical. At or above 1 equivalent of ATP, the reactions proceeded no further than 60%. Below 1 equivalent of ATP, the reactions proceeded in proportion to the amount of ATP added (e.g. 0.05 eq. to ˜5%, 0.3 eq to ˜30%, etc.) At the highest concentrations of ATP, there were no detrimental effects on the extent of reaction.

Without wishing to be bound by any particular theory, it is postulated that the ternary complex of A:sphnt:B does not suffer from over adenylation of either B substrate or ligase. This outcome would potentially lead to the release of adenylated donor and deplete the pool of available oligo. Therefore, the range of ATP concentrations that can be used for these experiments is broad, and the standard condition for further experiments is arbitrarily set to 10 eq relative to donor B.

### EXAMPLE 3

### Ligase Concentration

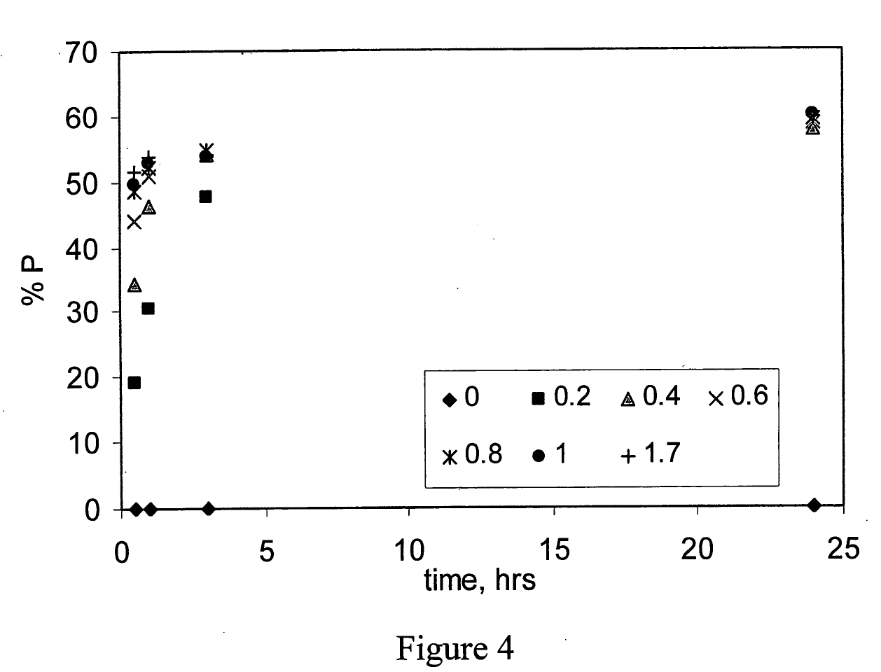

Ligation reactions were optimized as to ligase concentration, by varying the concentration of T4 RNA ligase. Using 10 eq. of ATP, ligations of A, B, and splint 21 were conducted with varying concentrations of ligase. Results are illustrated in FIG. 4.

As is evident, the initial velocity increases as a function of ligase concentration until 0.6 U/μL, at which point no additional gain is observed. The extent of reaction did not proceed beyond approximately 60%.

The conclusion from these results is that the concentration of enzyme can be set as standard at 0.8 U/μL to afford an adequate rate of reaction. This finding does not preclude the use of other concentrations of RNA ligase.

### EXAMPLE 4

### Annealing

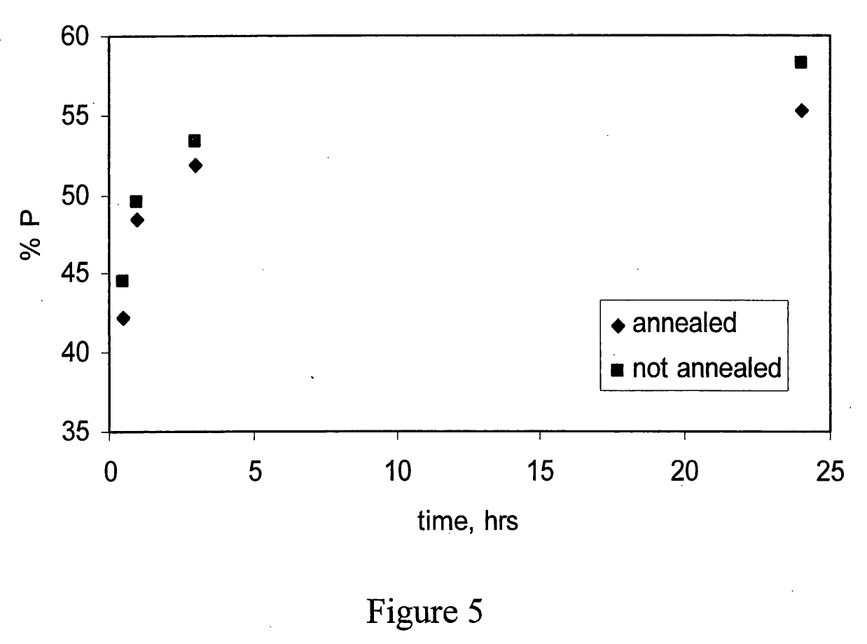

In order to determine if the extent of reaction observed was due to poorly annealed ternary complex, two ligations were performed with RNA oligos that either had or had not been annealed. Side-by-side reactions were conducted in 50 mM Tris.Cl, pH 7.8 (see FIG. 5). The first contained both A and B at the bottom of the tube, while the droplet of splint 21 was carefully placed inside and underneath the lid. In the second, all three RNA were simply added together. The first tube was then heated to 95° C. for 2 minutes before both tubes were separately mixed and spun-down, then mixed and spun-down again. In this manner, the RNA in the first tube was denatured without subjecting the splint and its labile ACE protecting groups to heat. Finally, the remaining reaction components and ligase were added to both tubes to initiate the reactions. Subsequent experiments in this report were performed without annealing the RNA oligos.

No effect of annealing is observed using this combination of RNA oligos. Note that the low ionic strength used in the annealing may prevent proper annealing. As is known in the art, proper annealing relies upon a combination of ionic strength and sequence dependency. Optimal annealing conditions for a given ionic strength using a given sequence can be readily determined by those skilled in the art. Subsequent experiments described herein below were performed without annealing the RNA oligos.

### EXAMPLE 5

### Splint Design

Initial results described above suggested that the hybridization strength of the B:splint pairing might influence the distribution of ligation products. A weaker B:splint pairing (expressed in terms of Tₘ, ° C.) was likely to yield the major product as the desired 41-mer, and three minor products, possibly concatamers of the B substrate.

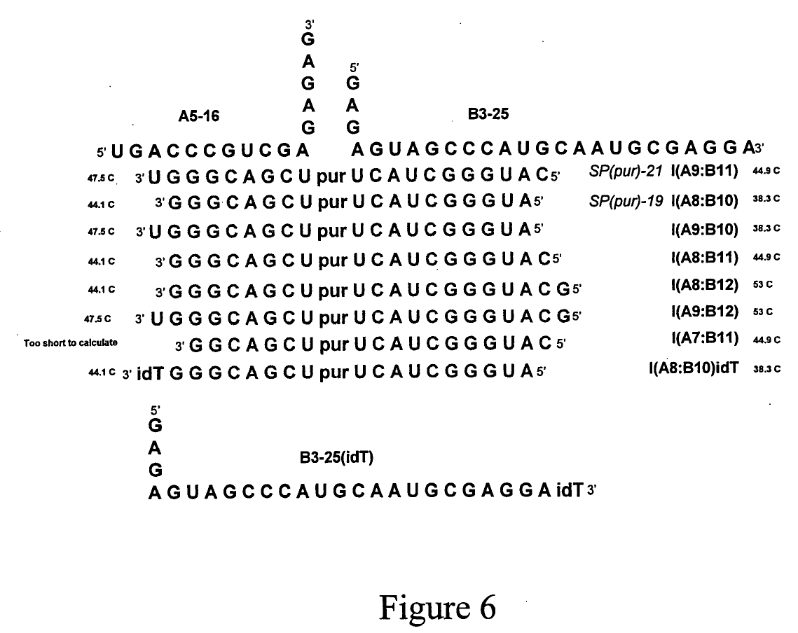

To test this hypothesis, the following splints were designed as shown in FIG. 6. Note that for standardization, the naming system for the splints was changed. By weakening or strengthening the A and B pairing to the splints, it was postulated that insight into the minimum hybridization strength could be gained. Furthermore, the 3′-blocked (idT) version of the 19-mer splint and the B substrate were made to determine if longer products were, in fact, concatamers of either B substrate or splint.

TABLE 2
Results of Modified Splints

| Splint | Tm A:Tm B | Result |
|---|---|---|
| I(A9:B11) | 48:45 | <1% undesired longer products |
| I(A9:B12) | 48:53 | <5% undesired longer products |
| I(A8:B10) | 44:38 | 34% undesired longer products |
| I(A8:B11) | 44:45 | Single desired product |
| I(A8:B12) | 44:53 | Single desired product |
| I(A7:B11) | n.d.:45 | <5% additional, longer products |
| I(A8:B10)idT | 44:38 | 34% Additional, longer products |
| I(A8:B10)idT + | 44:38 | Single desired product |
| (B3-25)idT | | |

These experiments established that a weak B:splint pairing would lead to multiple products. (Table 2) By lengthening the B:splint pairing by one or two bases from the original 19-mer I(A8:B10), to I(A8:B11) or I(A8:B12), the number of ligation products was reduced from four to one. Weakening A significantly with I(A7:B11) had no effect on product outcome. If both A and B were strengthened as in I(A9:B12), a very small fraction of longer products would result.

As concerns the nature of the longer products, when the original 19-mer splint was used with 3′-idT blocked B, the reaction yielded a single, desired product. 3′-blockage of the 19-mer splint alone yielded the distribution of four products as before. Therefore, the longer products observed with 19 and unblocked B are likely concatamers of the B substrate.

The conclusion from these results is that the Tₘ of the A and B:splint pairing are preferably at least 45° C. for the sequences employed. For other A and B:splint pairings with A's, B's and splints of different sequence composition than employed here, the procedures described above may be employed for optimization of Tm for A and B:splint pairings.

### EXAMPLE 6

### AMP or PPi Inhibition

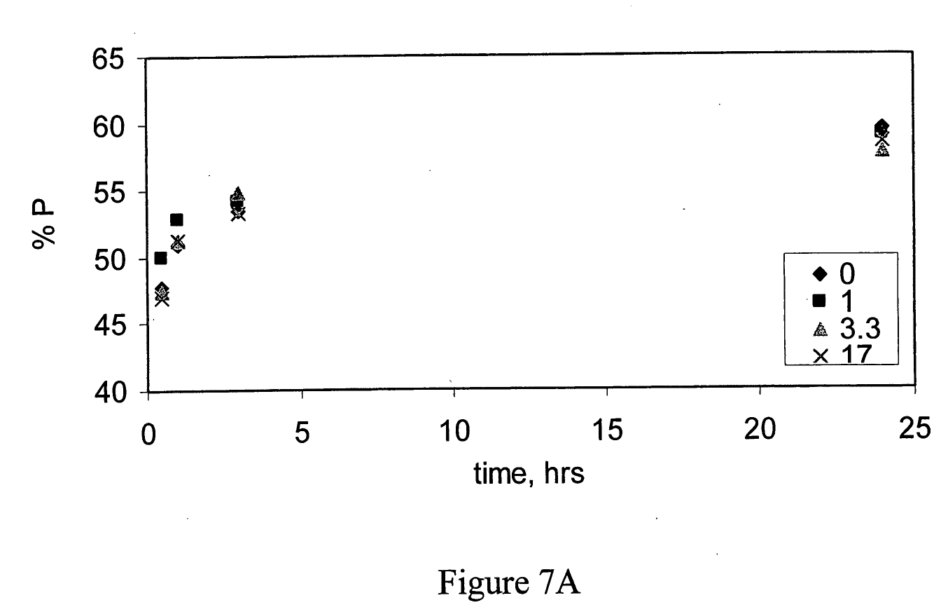

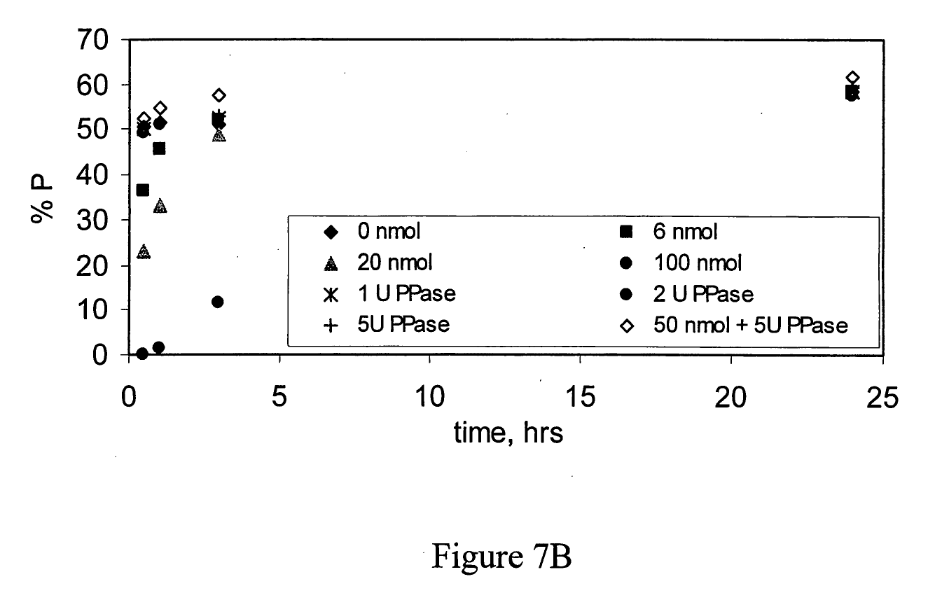

The ligase titration discussed above and shown in FIG. 4 reveals an increase in initial velocity as a function of enzyme concentration, However, the extent of the reaction plateaus at approximately 60% at all ligase concentrations. Typically, curves such as these imply product inhibition. To test this possibility, ligations were performed under standard conditions with 50 μM A oligo, 0.8 U/μL ligase, and 10 eq. ATP relative to B. In separate experiments, added AMP, inorganic pyrophosphate or pyrophosphatase were then added at varying concentrations (see FIGS. 7A and 7B).

These data show that AMP does not affect the outcome of the reaction. In contrast, inorganic pyrophosphate slows the rate but has no effect on the extent. Pyrophosphatase relieves the effect of added pyrophosphate, but has no apparent effect in standard, control reactions. Therefore, PPi is a product inhibitor and AMP is not. PPi release must only be rate limiting at very high concentrations, since no effect with (the extremely rapid) PPase is observed under normal conditions of less than 6 nmol PPi. This effect of PPi on the rate of reaction has been reported in the literature (Atencia, E. A., Madrid, O., Gunther Sillero, M. A., and Sillero, A. (1999) Eur J Biochem 261, 802-11; McLaughlin, L. W., Piel, N., and Graeser, E. (1985) Biochemistry 24, 267-73). An additional experiment that combines AMP and PPi with or without PPase may be of value. Further discussion of the extent of reaction is provided below in connection with experiments on 3′ blocking of donor RNA molecules and the use of 2′-ACE protection vs. naked, or 2′-OH, structure.

### EXAMPLE 7

### 3′ Block of Donor RNA Molecule

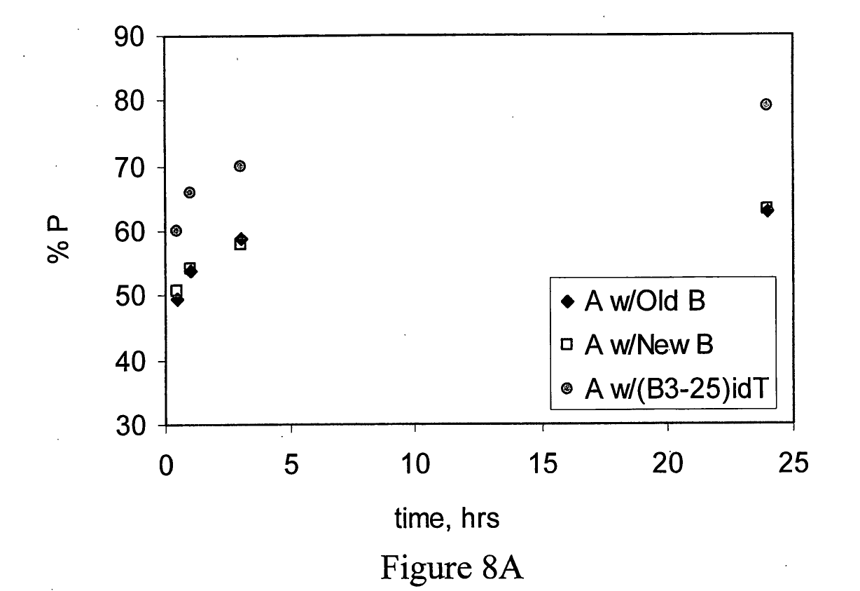

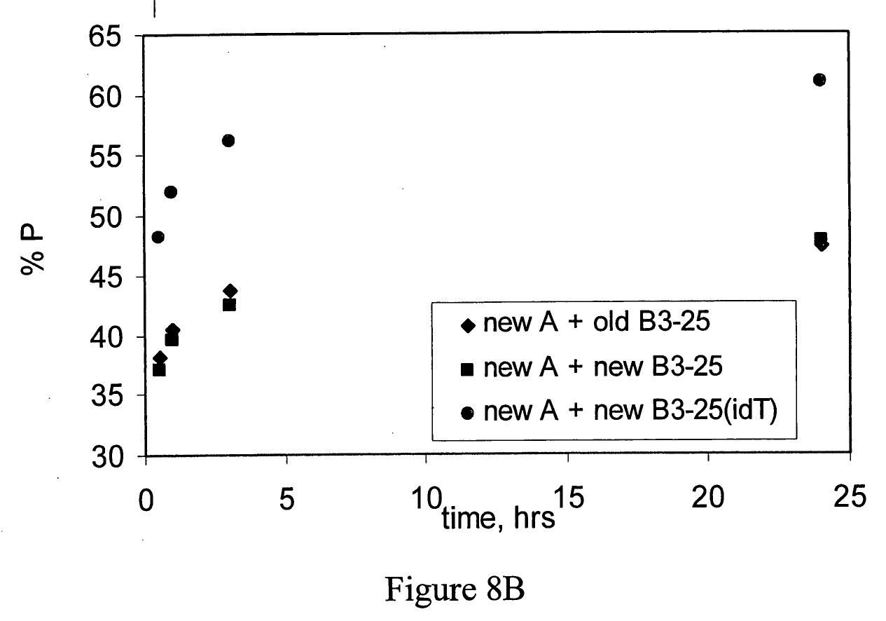

With the first ligation reactions to use the 3′-(idT)-B substrate, it was observed that the reaction proceeded further and more reliably than those ligating 3′-OH B. This is shown in FIGS. 8A and 8B. Furthermore, the integrity of the excess 3′-OH B substrate remaining in 24 hour timepoints is always much poorer than remaining 3′-idT B. One possibility is the substrate is degraded by the enzyme's inherent exonuclease activity (Krug, M., and Uhlenbeck, O. C. (1982) Biochemistry 21, 1858-64). However, this reaction requires a 3′-phosphate which the B substrate of these experiments does not include. As a control, several reactions of B and splint were incubated with reaction components and ligase for 24 hours to determine if contaminating nucleases were present in the commercial preparation. No degradation of B was observed under these conditions.

Upon examination of the 24 hour timepoints of ligations using 3′-OH B substrates, it was observed that a new peak (never greater than 5-10%) often appears running slightly longer than B. This peak does not form when using 3′-idT B, and requires that all ligation components are present including A and splint. The identity of this material has not been determined, but it is postulated that it represent a circularized B.

The conclusion from these results is that the B substrate can be consumed to give several outcomes. The major route is productive ligation to A to form the desired product. However, the extent of this reaction is often limited by formation of minor products and some degradation. Preliminary comparisons of 2′-OH vs. 2′-ACE suggest that complications introduced using 3′-blockage may be avoided by use of the 2′-ACE protected B. These results are discussed below.

### EXAMPLE 8

### 2′-ACE vs. 2′-OH

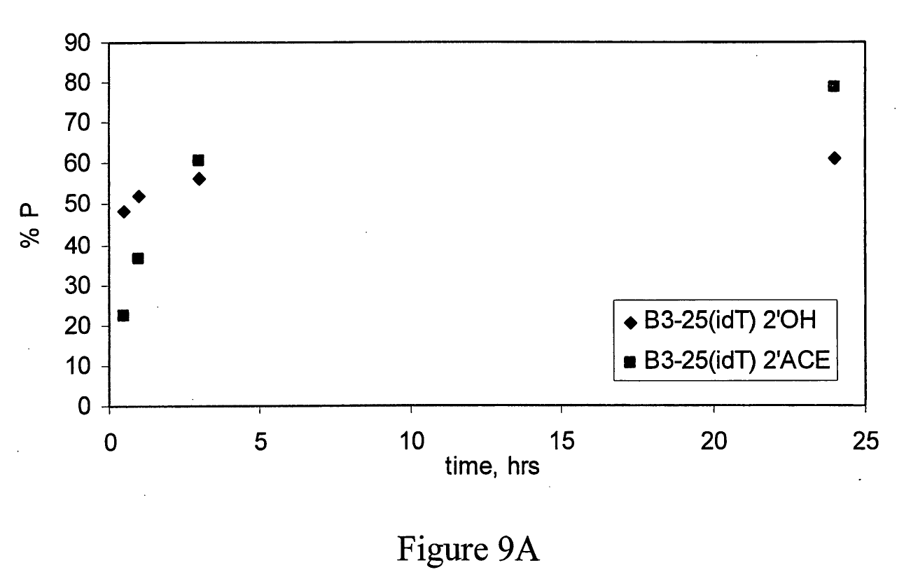

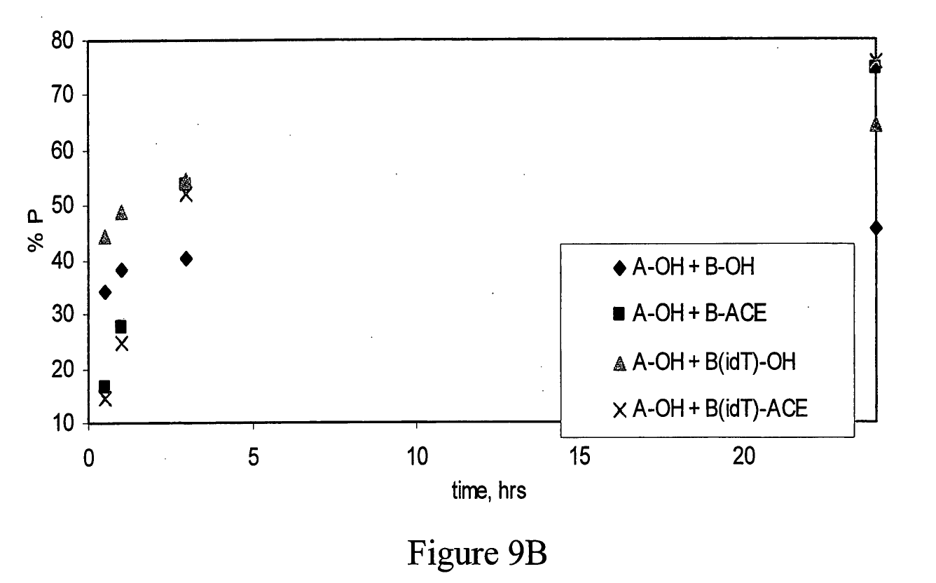

In order to reach oligo lengths beyond 120 bases, it will likely be necessary to perform sequential ligation reactions using oligos of 40-50 bases. Multiple rounds of ligations will reduce final yields significantly if individual reactions do not proceed beyond 60% as typically observed here. As 3′-idT blocked B substrates demonstrate, the reactions could proceed further if the non-productive consumption of B were to be reduced. Temporarily blocking the 3′ of B introduces several complications, however. Fortunately, an alternative strategy is suggested by work in prior investigations and confirmed by results reported here. For ligations to proceed, the A substrate may not be 2′-ACE protected, while the B substrate can be protected or in the native 2′-OH form. (See FIGS. 9A and 9B) Therefore, the reaction performed using 2′-OH A and 2′-ACE B with ACE protected splint proceeds without futile consumption of B and also much further than reactions using 2′-OH B.

### EXAMPLE 9

### Length of Ligation Linkers

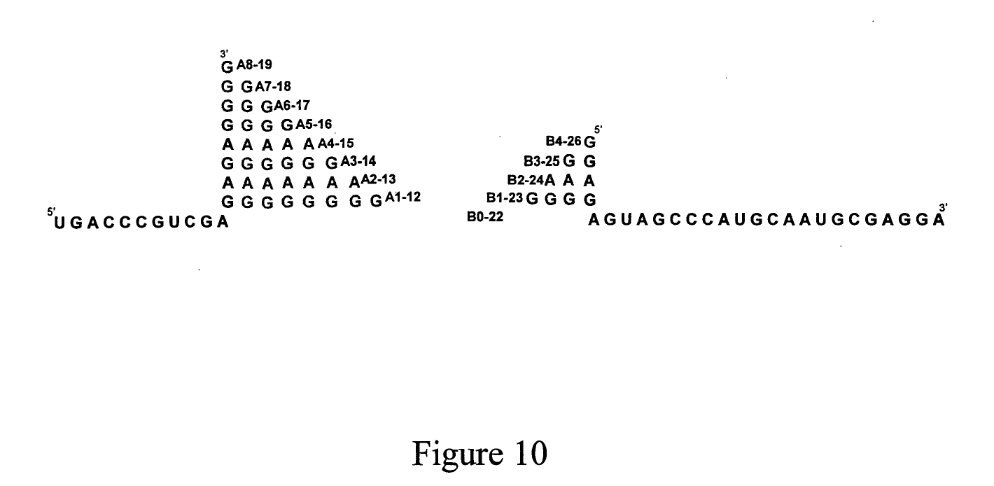

To determine if a 5 base ligation linker on A and 3 base ligation linker for B was truly optimal, an array of experiments was performed varying each. Beginning with all 2′-OH substrates and the I(A9:B11) splint, the length of the A single stranded ligation linker was varied from 8 down to 1, and ligated to B3-25. Then, the length of the B ligation linker was varied from 4 to 0, all ligated to A5-16 (see FIG. 10).

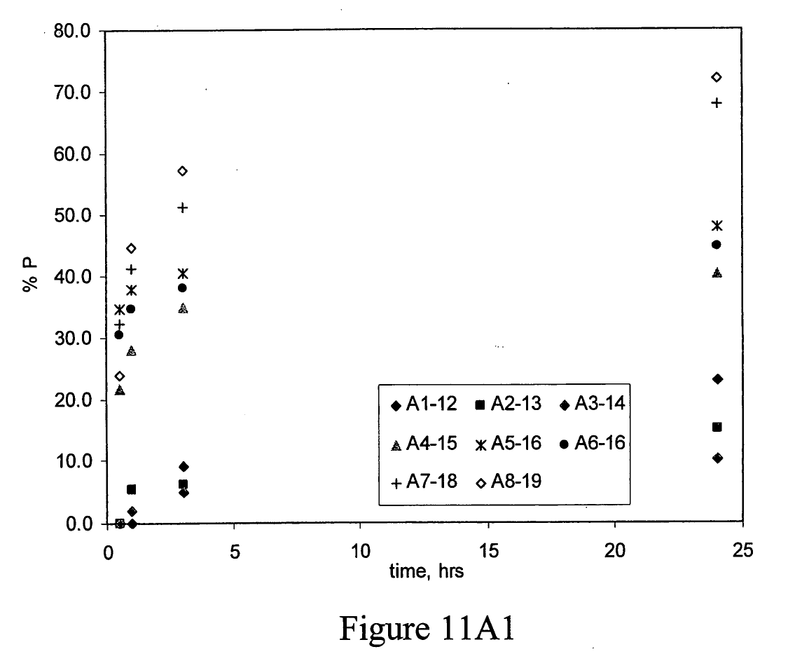

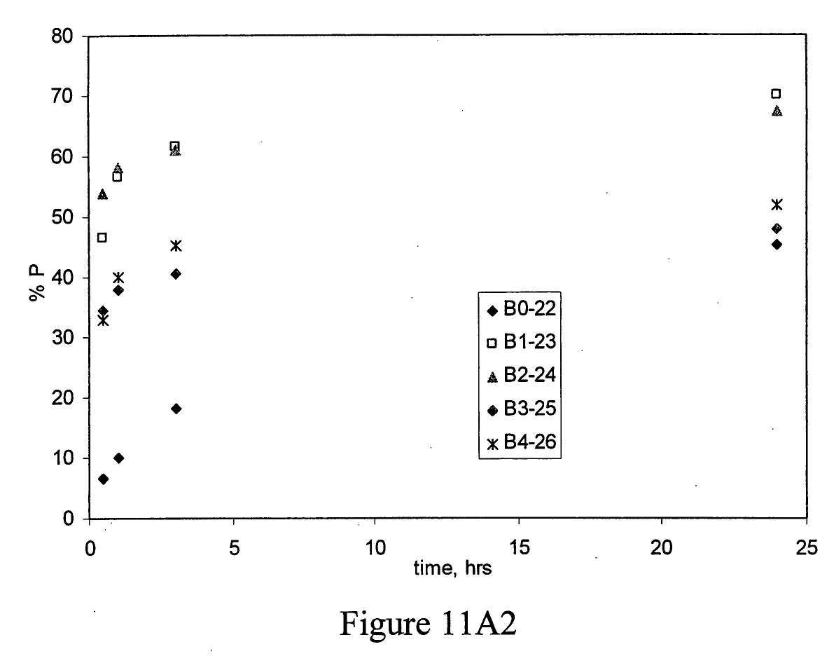

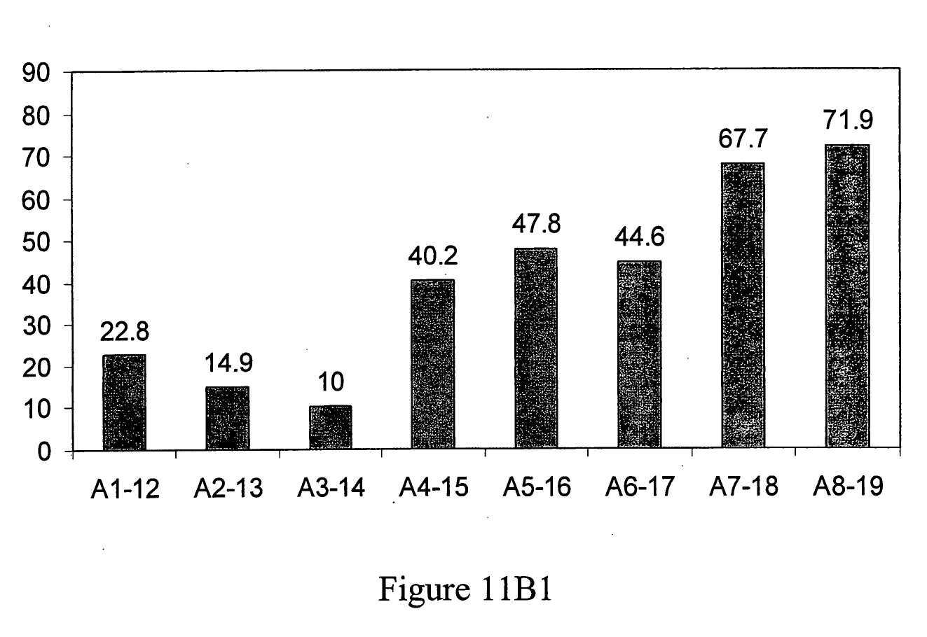

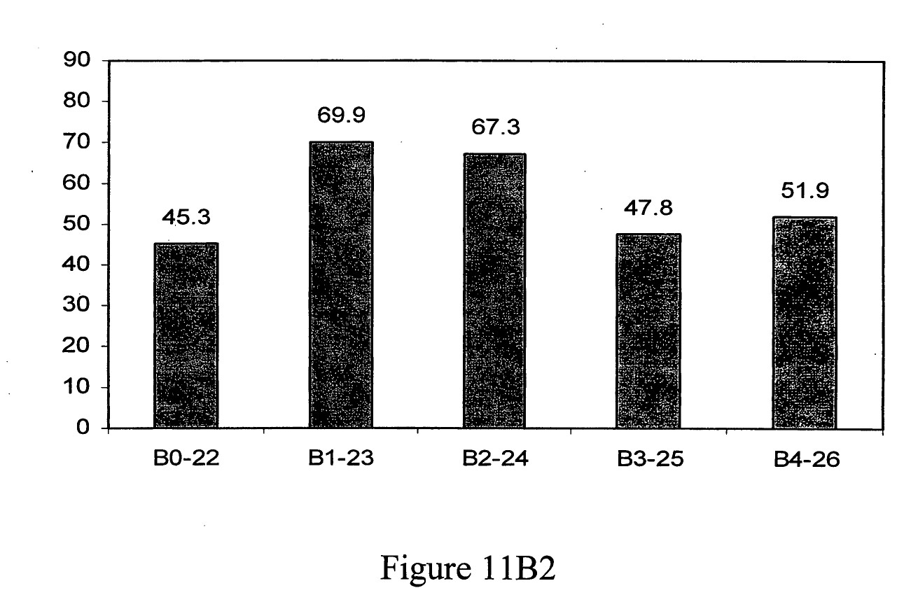

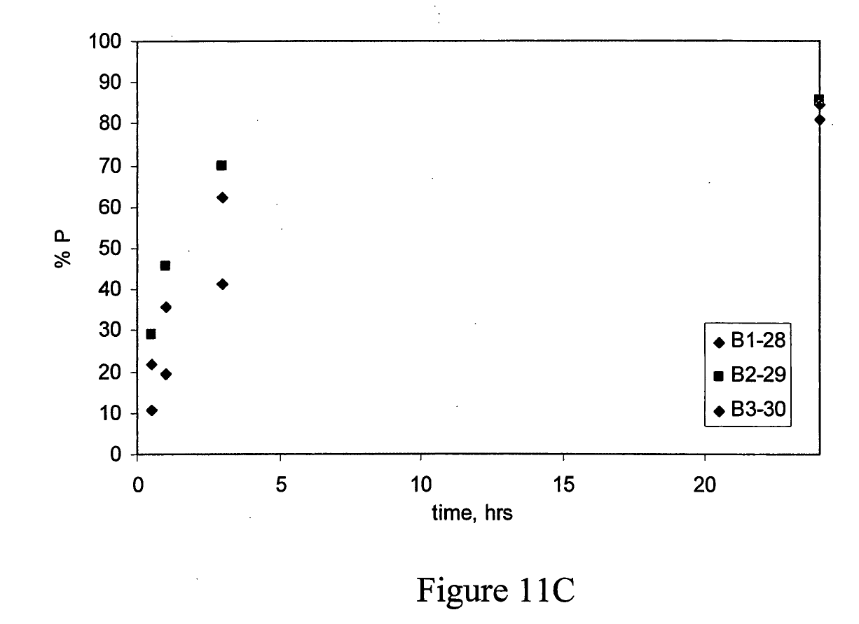

The results in FIGS. 11A1 and 11A2 show that the window of acceptable A ligation linkers is narrower than for B ligation linkers. For example, A ligation linkers served poorly as substrates unless they were at least 4 bases long. A5-16 through A8-19 were the best on the basis of rate and extent, but A7 and A8 tended to yield multiple, longer products. B ligation linkers were all substrates for ligation (see FIGS. 11B1 and 11B2). Surprisingly, B substrates with a ligation linker length of 1 or 2 actually proceeded further and faster than the standard B3-25.

The above experiment was repeated using 2′-OH A5-16 and the 2′-ACE B substrates with varying length ligation linkers. Analysis of these experiments was complicated by the fact that the ACE protected B0, B1, and B2 eluted at the same retention time as 2′-OH A5-16. Despite this, it was possible to determine that very little (<6%) ligated product was formed using the B0-22 substrate. To quantify the area of the A peak, (1) the time-points were deprotected and analyzed again, and (2) new B11, B2, and B3 substrates were designed adding 5 adenosines to the 3′-terminus (thereby lengthening their retention on HPLC) (see FIG. 11C).

Analysis of both the 2′-ACE vs. 2′-OH comparison and the ligation linker length variation experiments here reveal that the A ligation linker should have flexibility, while the B ligation linker should be rigid. 2′-ACE A oligonucleotides were not substrates, while 2′-ACE B substrates were. Much work at Dharmacon, Inc. has suggested that 2′-ACE protection prevents folding of oligonucleotides to form secondary and/or tertiary structure and maintains the RNA in a rigid, rod-like structure. Consistent with this observation, the shorter B ligation linkers used here should be more rigid, whereas the longer A ligation linkers are presumably more flexible. One possible explanation is that the phosphate of B must be precisely positioned for adenylation, while the ligation linker of A can then reach over to this activated species for reaction.

### EXAMPLE 10

### Ligation Linker Composition

Early work on T4 RNA ligase determined that the primary sequence of the bases at the splice site could be varied slightly. Work with nucleotides indicated that 5′-pC was the best donor, but the other purines and pyrimidines also worked.(McLaughhn, L. W., Piel, N., and Graeser, E. (1985) Biochemistry 24, 267-73) All sequences can serve as the 3′-terminal base of the acceptor (England, T. E., Gumport, R. I., and Uhlenbeck, O. C. (1977) Proc Natl Acad Sci USA 74, 4839-42).

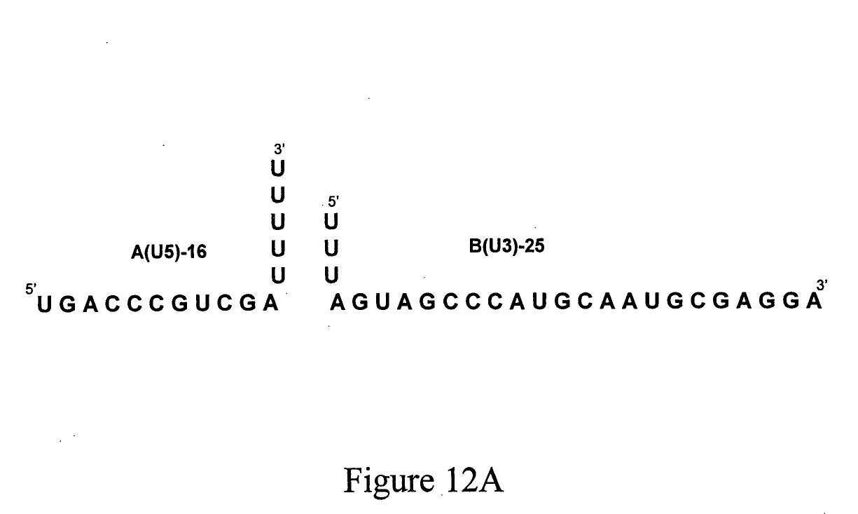

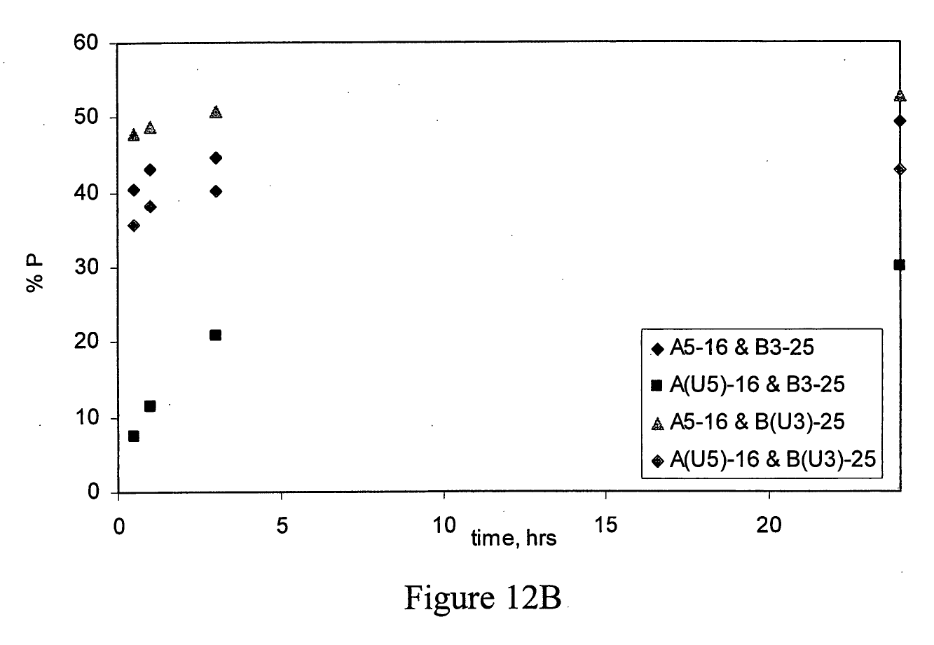

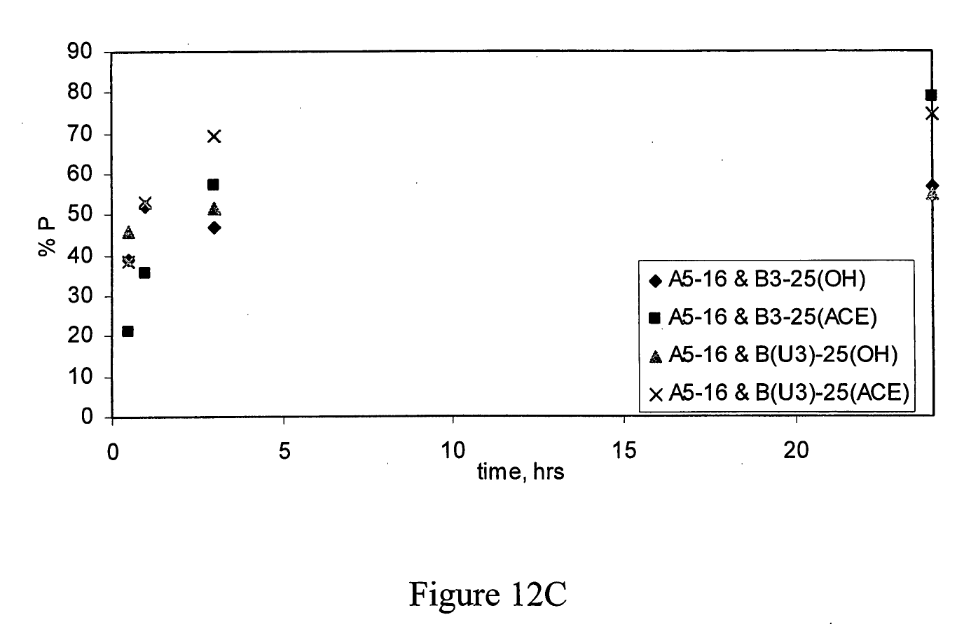

It was postulated that the enzyme would not differentiate the primary sequence of oligonucleotides in the ternary complex used here. To confirm this, two new substrates were designed with only pyrimidines in the ligation linkers. (FIG. 12A) These were then each tested with the complimentary A5-16, B3-25, or ligated to each other. It was recognized at this point that the poly-U ligation linker of the A substrate could anneal to the normal B ligation linker with a two base overhang, and that the poly-U ligation linker of the B substrate could anneal to the normal A ligation linker.

The results of this experiment, shown in FIGS. 12B and 12C, are not easily interpreted. If the last base of the A substrate can not be a pyrimidine, the A(U5)-16 to B(U3)-25 ligation should not have proceeded. If hybridization of the ligation linkers inhibits the reaction, then the A5-16 to B(U3)-25 ligation should not have proceeded. Only the A(U5)-16 to B3-25 ligation appears to have been affected by substitution of the urines in the standard ligation linkers. If the A(U5)-16 substrate was ligated to B substrates B1-22, B2-23, and B3-24, whose ligation linkers were presumably unable to hybridize with the ligation linker U's of A, the reaction proceeded normally. However, a less ambiguous experiment would be to substitute only the 3′-terminal residue bf A, and the 5′-base of B with a uracil. Although not shown conclusively, paired ligation linkers should be avoided. Fortunately, the sequence requirement of the ligation linker may not be very stringent.

### EXAMPLE 11

### RNA Concentration

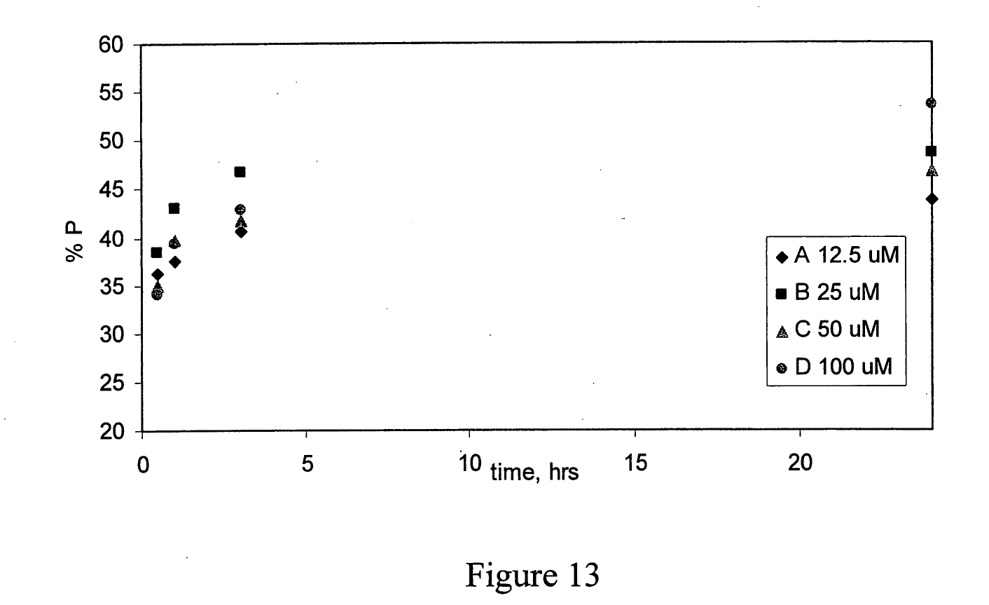

The standard RNA concentration for the above experiments is based on the amount of A substrate used in the reaction. Generally, the final concentration of A is held constant at 50 μM. One possibility to explain the fact that these reactions do not proceed beyond 90% might be that the oligonucleotide products are not readily released from the enzyme. Alternatively, the conditions of the reaction (i.e., pH, ionic strength) may be changing with time or consumption of substrate. To test this, four reactions were initiated with 4 different concentrations of substrates. In all, the ratio of 1:1.25:1.5 for A to splint to B was maintained. The concentration of all cofactors and ligase was held constant as per the standard. The concentration of A was 12.5, 25, the standard 50, and 100 μM. (FIG. 13) In all experiments, the rate and extent of reaction was unaffected by the concentration of RNA in the range tested. This suggests that some flexibility is possible for production scale. In addition, turnover-dependent changes in the environment do not appear to be a factor in the limited extent of reaction.

### EXAMPLE 12

### Stoichiometry

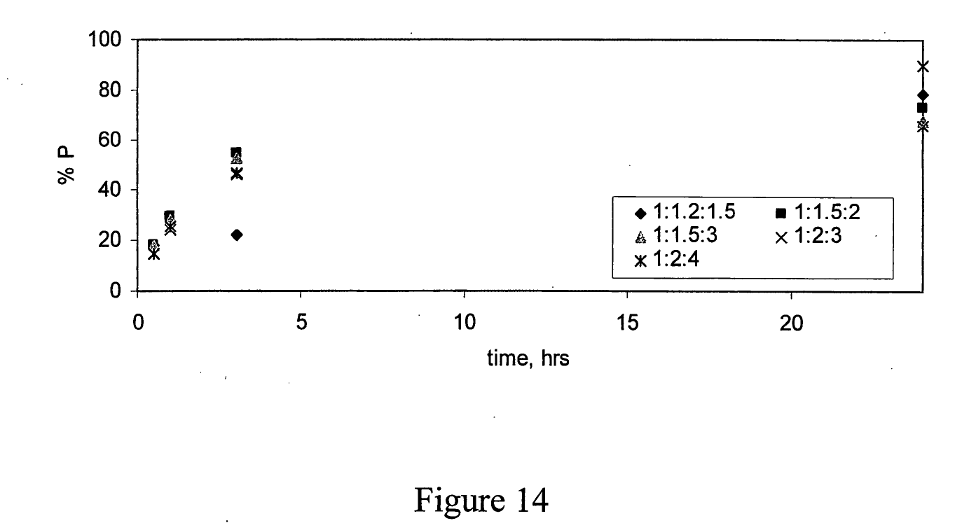

The stoichiometry of the A, B and spacer molecules was investigated. The ratio of the three RNA components in the ligation reaction was set at 1:1.25:1.5. To examine if variations of this ratio might promote the reactions going further to completion, several stoichiometries were investigated. (FIG. 14) In each, A5-16 was reacted with B3-25 in the ACE form using splint I(A9:B11). From these data, it appears that the reaction is insensitive to small changes in the relative amount of each oligo.

### EXAMPLE 13

### Optimized Ligation Conditions

Although RNA ligase reactions were originally thought to be very sensitive to several factors, especially ATP concentration and RNA substrate concentration, by carefully and methodically varying these parameters, the inventors have determined that these parameters can be varied without negative effects. The splint and downstream (donor) substrate can remain 2′-ACE protected, while the upstream (acceptor) substrate must be deprotected. There are several technical advantages for keeping the splint and donor substrate protected, beyond the obvious fact that both will be less susceptible to nucleases. These advantages include: (1) intermolecular hybridization is promoted by 2′-ACE protection, which inhibits intramolecular tertiary structures; and (2) side reactions are reduced, presumably because 2′-ACE donor molecules cannot participate in concatamerization or cyclicization reactions. In short, the 2′-ACE moiety uniquely allows for technological advances in ligation technology, and is significantly improved over any other method available.

In the above described experiments, reactions proceeded to approximately 80-85% completion. Some reasons investigated here include classical product inhibition, nuclease activity on the part of the ligase, and oligonucleotide stoichiometry. Even though a single product is produced, low yields would severely reduce the effectiveness of sequential ligation strategies.

As a result of the experiments described above, a set of preferred conditions for splint assisted RNA ligation have been compiled, and are listed below.
Deprotected acceptor substrate (1 equiv., 50 μM) with 5-6 bases unpaired 3′.
ACE Protected acceptor substrate (1.5 equiv.) with phosphate and 1 base unpaired 5′.
ACE Protected splint (1.25 equiv.) with purine spacer opposite ligation site.
10 equivalents ATP (750 μM)
50 mM Tris
10 mM DIT and MgCl₂.
Ambient temperature.
0.8 U/μL Ligase

Once synthesized, the ligation products of the present invention may immediately be used or be stored for future use. Preferably, the ligation products of the invention are stored as duplexes in a suitable buffer. Many buffers are known in the art suitable for storing RNAs. For example, the buffer may be comprised of 100 mM KCl, 30 mM HEPES-pH 7.5, and 1 mM MgCl₂. Preferably, the double stranded polynucleotides of the present invention retain 30% to 100% of their activity when stored in such a buffer at 4° C. for one year. More preferably, they retain 80% to 100% of their biological activity when stored in such a buffer at 4° C. for one year. Alternatively, the compositions can be stored at −20° C. in such a buffer for at least a year or more. Preferably, storage for a year or more at −20° C. results in less than a 50% decrease in biological activity. More preferably, storage for a year or more at −20° C. results in less than a 20% decrease in biological activity after a year or more. Most preferably, storage for a year or more at −20° C. results in less than a 10% decrease in biological activity.

In order to ensure stability of the RNA prior to usage, they may be retained in dried-down form at −20° C. until they are ready for use. Prior to usage, they should be resuspended; however, even once resuspended, for example, in the aforementioned buffer, they should be kept at −20° C. until used. The aforementioned buffer, prior to use, may be stored at approximately 4° C. or room temperature. Effective temperatures at which to conduct transfection are well known to persons skilled in the art, but include for example, room temperature.

Although the invention has been described and has been illustrated in connection with certain specific or preferred inventive embodiments, it will be understood by those of skill in the art that the invention is capable of many further modifications. This application is intended to cover any and all variations, uses, or adaptations of the invention that follow, in general, the principles of the invention and include departures from the disclosure that come within known or customary practice within the art and as may be applied to the essential features described in this application and in the scope of the appended claims.

1. A method of ligating ribonucleotides comprising:
ligating a donor RNA molecule to an acceptor RNA molecule in the presence of RNA ligase and a splint, wherein the donor RNA molecule is comprised of at least one nucleotide and a ligation linker moiety, the acceptor RNA molecule is comprised of at least one nucleotide and a ligation linker moiety and the splint is comprised of a polyribonucleotide.

2. The method of claim 1, wherein the donor RNA molecule further comprises at least one modified nucleotide.

3. The method of claim 2, wherein the at least one modified nucleotide is an orthoester modified nucleotide.

4. The method of claim 3, wherein the orthoester modified nucleotide comprises an orthoester moiety bonded to a 2′ carbon of said at least one nucleotide of the donor RNA.

5. The method of claim 4, wherein the orthoester modified nucleotide comprises an orthoester moiety bonded to the 2′ carbon of every nucleotide the donor RNA.

6. The method of claim 1, wherein the splint further comprises at least one modified nucleotide.

7. The method of claim 6, wherein the at least one modified nucleotide is an orthoester modified nucleotide.

8. The method of claim 7, wherein the orthoester modified nucleotide comprises an orthoester moiety bonded to a 2′ carbon of said at least one nucleotide of the splint.

9. The method according to claim 1, wherein the splint comprises an orthoester moiety bonded to the 2′ carbon of each nucleotide of the splint.

10. The method of claim 1, wherein said ligating proceeds to at least 80% completion.

11. The method of claim 10, wherein said ligating proceeds to at least 85% completion.

12. The method according to claim 1, wherein the splint further comprises a spacer.

13. The method according to claim 16, wherein the spacer is comprised of purines.

14. The method of claim 1, wherein the acceptor RNA molecule further comprises at least one modified nucleotide.

15. The method according to claim 18, wherein the at least one modified nucleotide is an orthoester modified nucleotide.

16. The method according to claim 1, wherein the acceptor RNA molecule has an orthoester moiety bonded to the 2′ carbon of each ribosyl moiety.

17. The method according to claim 1, wherein the acceptor RNA molecule further comprises a 3′terminal —OH.

18. The method according to claim 1, wherein the donor RNA molecule further comprises a 5′ terminal phosphate moiety.

19. The method according to claim 1, wherein the length of the ligation linker of the acceptor RNA is greater than or equal to four nucleotide bases.

20. The method according to claim 1, wherein the length of the ligation linker of the donor RNA molecule is greater than or equal to two nucleotide bases.

21. The method according to claim 25, wherein the length of the ligation linker of the donor RNA molecule is eleven to twelve nucleotide bases.

22. The method according to claim 1, wherein the donor RNA molecule and the acceptor RNA molecule each have a Tm with respect to the splint of at least 45 degrees Centigrade.

23. The method according to claim 1, wherein said ligating is carried out in the presence of a pyrophosphatase.

24. The method according to claim 1, wherein the acceptor RNA molecule further comprises a first modified nucleotide, and the donor RNA molecule comprises a second modified nucleotide that is different from the first modified nucleotide.

25. The method according to claim 1, wherein the splint comprises a polyribonucleotide having a blocked 3′ terminus.

26. A splint for use in ligating RNA molecules, comprising:
a polyribonucleotide, wherein said polyribonucleotide has at least one orthoester modified nucleotide base.

27. The splint according to claim 31, further comprising a spacer.

28. The splint according to claim 31, wherein the at least one orthoester modified nucleotide base is a 2′ orthoester modified nucleotide base.
